(19) United States
(12) Patent Application Publication
(10) Pub. No.: US 2024/0260738 A1
(43) Pub. Date: Aug. 8, 2024

(54) HAIRCARE MONITORING AND FEEDBACK

(71) Applicant: Conopco, Inc., d/b/a UNILEVER, Englewood Cliffs, NJ (US)

(72) Inventors: Anthony BROWN, Nottingham (GB); Paul John CUNNINGHAM, Carnforth (GB); Robert Lindsay TRELOAR, Hoole (GB); Michel François VALSTAR, Nottingham (GB)

(21) Appl. No.: 18/291,397

(22) PCT Filed: Jul. 22, 2022
(86) PCT No.: PCT/EP2022/070633

§ 371 Date: Jan. 23, 2024

(30) Foreign Application Priority Data
Jul. 29, 2021 (EP) 21188571.0

(51) Int. Cl. A46B 15/00; A45D 24/00

(52) U.S. Cl. CPC A46B 15/0006; A45D 24/00; A46B 15/004; A46B 2200/104

(57) ABSTRACT

A computer-implemented method for generating feedback for a user performing haircare includes receiving a sequence of images of a head of the user and a haircare implement, the haircare implement being a hairstyling implement. The computer-implemented method also includes determining one or more implement motion parameters by tracking a position or an orientation of the haircare implement using the sequence of images. The computer-implemented method also includes determining a detangling event if one or more of: linear velocity, angular velocity, linear acceleration, and angular acceleration of the haircare implement is less than a corresponding detangling motion threshold, the detangling event being an event in which tangles from hair of the user are being removed. The computer-implemented method also includes generating feedback for providing to the user based on the determined detangling event.

### FIELD

The present disclosure relates to a system and method for assisting a user performing personal grooming and in particular, although not exclusively, for assisting a user performing hairstyling.

### BACKGROUND

The effectiveness of a person's haircare routine can vary considerably according to a number of factors including the duration of the haircare routine, the skill of the stylist, the condition of the hair and the haircare technique. A number of systems have been developed for tracking the motion of a hairbrush adjacent to a user's head in order to provide feedback on brushing technique and to assist the user in achieving an optimum haircare routine.

Some of these brush tracking systems have the disadvantage of requiring motion sensors such as accelerometers built into the hairbrush. Such motion sensors can be expensive to add to an otherwise low-cost and relatively disposable item such as a hairbrush and can also require associated signal transmission hardware and software to pass data from sensors on or in the brush to a suitable processing device and display device.

It would be desirable to be able to monitor a user's haircare routine, such as tracking the motion of a brush or other haircare appliance adjacent to a user's head, without requiring electronic sensors to be built in to, or applied to, the hairbrush itself. It would also be desirable to be able to monitor a user's haircare routine using a relatively conventional video imaging system such as that found on a ubiquitous ‘smartphone’ or other widely available consumer device such as a computer tablet or the like. It would be desirable if the video imaging system to be used need not be a three-dimensional imaging system such as those using stereoscopic imaging. It would also be desirable to provide a brush or other care appliance tracking system which can provide a user with real-time feedback based data obtained during a haircare session. Some aspects of the present disclosure may achieve one or more of the above objectives.

### SUMMARY

According to a first aspect of the present disclosure, there is provided a method for assisting a user in performing haircare, the method comprising:
receiving a sequence of images of a head of the user and a haircare implement;
determining one or more implement parameters by tracking a position and/or orientation of the haircare implement using the sequence of images; and
generating or providing feedback for the user based on the one or more implement parameters.

According to an example embodiment, the method may comprise determining one or more performance parameters and/or one or more haircare events based on the implement parameters. The method may comprise providing the feedback based on the one or more performance parameters and/or the one or more haircare events.

In one or more examples, the feedback may comprise instructions for the user in how to perform the haircare or a recommendation of an appropriate chemical or heat treatment including but not limited to particular product recommendations.

In one or more examples, the one or more haircare events may include one or more of: a detangling event, an inadequate implement stroke and an inadequate implement-hair contact.

In one or more examples, the one or more performance parameters may include one or more of: an applied implement force, an implement-hair grip and a user satisfaction.

In one or more examples, tracking a position and orientation of the haircare implement may comprise tracking the position and orientation relative to the head of the user.

In one or more examples, tracking the position and orientation relative to the head of the user may comprise tracking the position and orientation relative to one or more face landmarks.

In one or more examples, tracking the position and orientation of the haircare implement may comprise tracking a fiducial marker coupled to an end of the haircare implement.

In one or more examples, the fiducial marker may form an integral part of the haircare implement or comprise an attachment coupled to the haircare implement.

In one or more examples, the fiducial marker may comprise a substantially spherical marker having a plurality of coloured quadrants disposed around a longitudinal axis corresponding to a longitudinal axis of the haircare implement.

In one or more examples, each of the quadrants may be separated from an adjacent quadrant by a band of strongly contrasting colour. The band of strongly contrasting colour may be highly contrasting with the colour of just one of the adjacent quadrants or may be highly contrasting with the quadrant either side of the band.

In one or more examples, the implement parameters may include one or more of: implement position; implement orientation; linear velocity; angular velocity; linear acceleration; angular acceleration and implement path.

In one or more examples, the haircare implement may comprise one of a hairbrush, a comb, a detangling comb/brush or any other hair styling implement. The method may further comprise determining a detangling event if one or more of: linear velocity, angular velocity, linear acceleration and angular acceleration are less than a corresponding detangling motion threshold.

In one or more examples, a detangling event may be determined if one or more of: linear velocity, angular velocity, linear acceleration and angular acceleration are substantially equal to zero.

In one or more examples, the method may also comprise determining an inadequate implement stroke based on an implement path.

In one or more examples, the implement path may be determined based on the position of the fiducial marker over a series of images.

In one or more examples, determining an inadequate implement stroke may be performed by a machine learning algorithm. The machine learning algorithm may comprise a relational model between implement path and hair outcome.

In one or more examples, determining an inadequate implement stroke may also comprise identifying an implement stroke associated with hair which is more difficult to manage. This may be due to the hair being unhealthy or hair which is naturally more prone to tangling. For example, it may be due to the hair being wavy or curly which may naturally be more difficult to manage.

In one or more examples, the method may further comprise identifying a suitable chemical treatment in accordance with a detected performance parameter or haircare event. Providing the feedback may comprise providing instructions to the user to perform the identified chemical treatment.

In one or more examples, identifying a suitable chemical treatment may comprise identifying a formulated product or an amount of time a formulated product is to be used for.

In one or more examples, providing feedback may comprise providing instructions for the user to operate the haircare implement in a particular way.

In one or more examples, the instructions may be provided during or after the user is performing haircare. The user may perform the haircare in a session.

In one or more examples, the method may further comprise identifying an implement technique in accordance with a detected performance parameter or haircare event.

Providing the feedback may comprise providing instructions to the user to perform the implement technique.

In one or more examples, the implement technique may comprise an implement path, an implement position, an implement orientation and/or an applied implement force. In one or more examples, the applied implement force may be determined by monitoring one or more of the linear velocity, angular velocity, linear acceleration and angular acceleration of the implement, or any other parameter that could be a proxy for force applied.

In one or more examples, the method may comprise determining a facial expression of the user in the sequence of images. The method may comprise determining the one or more performance parameters and/or the one or more haircare events based on the facial expression of the user and the implement parameters.

In one or more examples, the facial expression may comprise expressing pain. The one or more haircare events may include one or more of a detangling event and an inadequate implement-hair contact. The one or more performance parameters may comprise one or more of an applied implement force and an implement-hair grip.

In one or more examples, the facial expression may comprise or indicate one or more apparent emotional responses including, for example, apparent happiness, apparent pain, and apparent confidence. The one or more performance parameters may comprise a user satisfaction.

In one or more examples, the haircare implement may comprise: a hairbrush; a comb; curling tongs; hair straighteners; or a user's hand.

In one or more examples, the method may comprise analysing a haircare performance by determining a facial expression of the user in the sequence of images. The method may comprise providing feedback for the user based on the haircare performance.

In one or more examples, the method may be a computer-implemented method.

According to a further aspect of the present disclosure there is provided a haircare monitoring system comprising a processor configured to:
receive a sequence of images of a head of the user and a haircare implement;
determine one or more implement parameters by tracking a position and/or orientation of the haircare implement using the sequence of images; and
provide feedback for the user performing haircare based on the one or more implement parameters.

Also disclosed is a method of tracking a consumers hair grooming and hair styling activity receiving video images of consumers face and hair and using machine learning in combination with a brush with tracker attachment to track hair detangling/grip during styling and predict key hair damage and hair shape consumer benefits.

Also disclosed is a method of tracking a consumers hair grooming and styling activity receiving video images of consumers face and hair and using machine learning in combination with a brush with a tracker attachment to measure facial expression during hair detangling/styling events and link facial expression (pain, grimace) to consumer hair grooming routine/damage level making product recommendations.

Also disclosed is a method for tracking a consumer's hair style in real time receiving video images and using machine learning to link hair movement to fixed facial features to predict natural movement and make product recommendations.

There may be provided a computer program, which when run on a computer, causes the computer to configure any apparatus, including a circuit or device disclosed herein or perform any method disclosed herein. The computer program may be a software implementation, and the computer may be considered as any appropriate hardware, including a digital signal processor, a microcontroller, and an implementation in read only memory (ROM), or electronically erasable programmable read only memory (EEPROM), as non-limiting examples.

The computer program may be provided on a computer readable medium, which may be a physical computer readable medium such as a disc or a memory device, or may be embodied as a transient signal. Such a transient signal may be a network download, including an internet download. There may be provided one or more non-transitory computer-readable storage media storing computer-executable instructions that, when executed by a computing system, causes the computing system to perform any method disclosed herein.

A further aspect of the disclosure relates to a computer system. The computer system may be provided by a user device, such as a mobile telephone or tablet computer. The images may be received by a processor of the computer system from a camera of the computer system. In some examples, all processing of user images may be performed locally to improved user privacy and data security. In this way, it can be ensured that images of the user never leave the user's phone. Captured images may be deleted once processed.

Analysis of the captured images may be transmitted from the user device to a remote device.

### BRIEF DESCRIPTION OF THE DRAWINGS

One or more embodiments will now be described by way of example only with reference to the accompanying drawings in which:

FIG. 1 illustrates a haircare monitoring system;

FIG. 2 illustrates a method of assisting a user with a haircare routine;

FIG. 3 illustrates an example marker for a personal care implement;

FIG. 4A illustrates the marker of FIG. 3 coupled to a hairbrush;

FIG. 4B illustrates the marker of FIG. 3 coupled to another hairbrush in use;

FIG. 5 illustrates example hair styles;

FIG. 6 illustrates a method of detecting a detangling event;

FIG. 7 illustrates study data collected from a haircare session monitored via a smartphone camera;

FIG. 8A illustrates a first blown-up portion of the profiles illustrated in FIG. 7;

FIG. 8B illustrates a second blown-up portion of the profiles illustrated in FIG. 7;

FIG. 8C illustrates a third blown-up portion of the profiles illustrated in FIG. 7;

FIG. 9 illustrates further study data captured according to a specific training protocol;

FIG. 10 shows plots of pain scores against various force brush parameters for data captured according to the protocol of FIG. 9;

FIG. 11 shows plots of pain scores against various motion parameters determined by the brush motion classifier based on the marker position and orientation in the image data;

FIG. 12 illustrates the data of FIG. 11 after filtering together with additional marker derived data for angular speed;

FIG. 13 illustrates the x-position plotted against the y-position of the data points of FIG. 11 with associated pain scores;

FIG. 14 illustrates a correlation matrix identifying correlation strength between various parameters of the data for the force brush and the marker brush;

FIG. 15 shows plots of pain scores against various motion parameters determined by the brush motion classifier based on the marker position and orientation in the image data for hair styling segment data;

FIG. 16 illustrates the speed data of FIG. 15 after filtering;

FIG. 17 illustrates the x-position plotted against the y-position of the data points of FIG. 15 with their associated pain scores;

FIG. 18 illustrates the dependence of applied brush force on formulated product choice; and

FIG. 19 illustrates an example method of a haircare monitoring system according to an embodiment of the present disclosure.

### DETAILED DESCRIPTION

The disclosed system may provide a hair grooming and styling behaviour tracking system. In general terms, the system may make use of one or both of: (i) a 3D motion tracking component based upon tracking a known marker on a haircare implement; and (ii) a facial muscle/landmark tracking component which is used to track face position/poise, and a measure of apparent emotion (pain, happiness etc).

Apparent emotion is that which can be inferred from facial expression—via a facial expression model which takes as input movements of particular-points on Facial Muscle Activation Units (FAUs). The facial expression model can be trained prior to use for a given emotion using a panel of test subjects. Emotion recognition from measurements may be referred to as “Affective Computing.”

Apparent (or expressed) emotion may not directly reflect the emotion that an individual feels. For example, users may express pain to a differing degree. However, the expressed emotion measurement can be comparable across subjects/users and therefore provides an automatically calibrated parameter.

In one example, the user may be asked to provide their views on the level of pain they experience during a haircare session. The feedback may be provided, for example, by the user providing input indicating how painful an event or an overall session was on a scale, such as 0-10. The user input may then be compared with a corresponding apparent pain score. A plurality of such comparisons may be determined in order to create a correction value for the user. For example, if the user input pain value is 5 and a corresponding apparent pain value is 8 then the difference value between the values is +3. The mean of the differences for a plurality of such comparisons may be used as a correction value for calibration purposes. The plurality of sets of inputs may be obtained within a single haircare session or across a number of different haircare sessions. In the case that the sets of inputs are obtained in different sessions over an extended period of time, a weighting may be applied so that more recent sessions are given prominence in case the user's pain response has changed over time.

Calibrating the system allows the subsequent pain values determined by the system to more accurately reflect the user's experience, which may allow more relevant feedback to be provided to the user.

In addition, in a product testing environment, calibration procedures such as that discussed above may be used to standardize the responses between participants. Alternatively, such calibration procedures may allow users with a similar pain response to be selected as part of a group to assess a product. It will be appreciated that product testing in this context may relate to a chemical treatment for the user or their hair, or to a haircare implement, for example.

Both motion (brush tracking) and emotion (face emotion tracking) components work off the received sequence of images of the hair grooming and styling process which can be collected using a mobile device. In some examples, the system can exploit the sensing and compute power of modern mobile phones (or tablet, other edge computing devices) as it does not require any sensing capability external to the phone and all image processing can be performed on the device, enabling real-time feedback to the user. As such, the user can use their existing hardware, upgraded with bespoke software, and possibly in combination with a provided known marker, to assist in self-monitoring their haircare routine.

Many people (users) wash their hair several times a week (typically 2-5 times a week, sometimes daily). The hair washing routine typically includes use of a shampoo. The hair washing routine may also include the use of a second product—a conditioner product. These ‘rinse-off’ products are applied during the showering routine and rinsed out before the end of the washing process. A user may use a towel to dry excess moisture from the hair, prior to a hairstyling or haircare routine. The haircare routine may be divided into two sub-processes: a grooming process; and a styling process.

Many users will groom their hair prior to styling. The grooming process removes tangles from the hair (detangling process) using a brush or comb, which can be a painful and unpleasurable experience for the user.

The styling process typically follows one of two approaches:
Heat Styling—During a heat styling process, users can heat style their hair using a blow drier and a hairbrush. This may be followed by a further heat styling implement such as a straightening iron or curling tong. The heat styling is generally performed to ‘transform’ the hair shape from its natural shape into a different shape. Users may seek to adjust a volume of their hair, for example very straight hair (‘Volume Down’ or less volume than natural) or ‘Volume Up’ (more volume/body than natural). The shape transformation can be achieved through a ‘water wave’ with heat and tension applied to the hair as it goes from wet to dry (combination of blow drying or other heated haircare implement and a brushing/combing action). The force applied during the heat styling process can influence the outcome of the hair styling process, with an optimum level of force or grip on the brush or comb resulting in a desired outcome.
Natural Driers—Many users let their hair dry naturally. Following towel drying the user may only brush or comb out the tangles and then use a brush or even their fingers to style their hair, for example using their fingers to twist the hair into defined curls. These users are ‘shape enhancers’ rather than ‘shape transformers’. They may equally experience difficulties in detangling their hair.

Users may use a third product—a post-wash haircare product either before, during or after their haircare (hairstyling) routine, for example leave-on conditioner, gel, cream, mouse, serum, putty or Hairspray. Some of these products may be applied at the end of styling to ‘fix’ the style and make it last.

The rinse-off products used in the washing/treatment stage can have an important impact on the level of detangling experienced in the hair grooming process. A user may not easily connect the consequence of their product choice to an amount of detangling experienced. For example, users may use rinse off products with little or no silicone because they think it works best for hair styling or colour care.

The rinse off products used in the washing/treatment stage can also impact the hair styling process. For heat stylers, the amount of grip on the brush achieved can be impacted by the choice of shampoo and conditioner product. This ultimately impacts ability to achieve the desired end look (Volume up, straight, etc).

It would be desirable to provide a system that can monitor a user's haircare routine and provide feedback which can make the user's haircare experience less painful and/or more pleasurable and help the user better achieve their desired outcome. Beneficial feedback may include tips and advice and/or product recommendations for use during or at the end of the washing process and/or during the haircare (grooming/styling) routine (e.g. “you are brushing too hard”; “next time, be more gentle”).

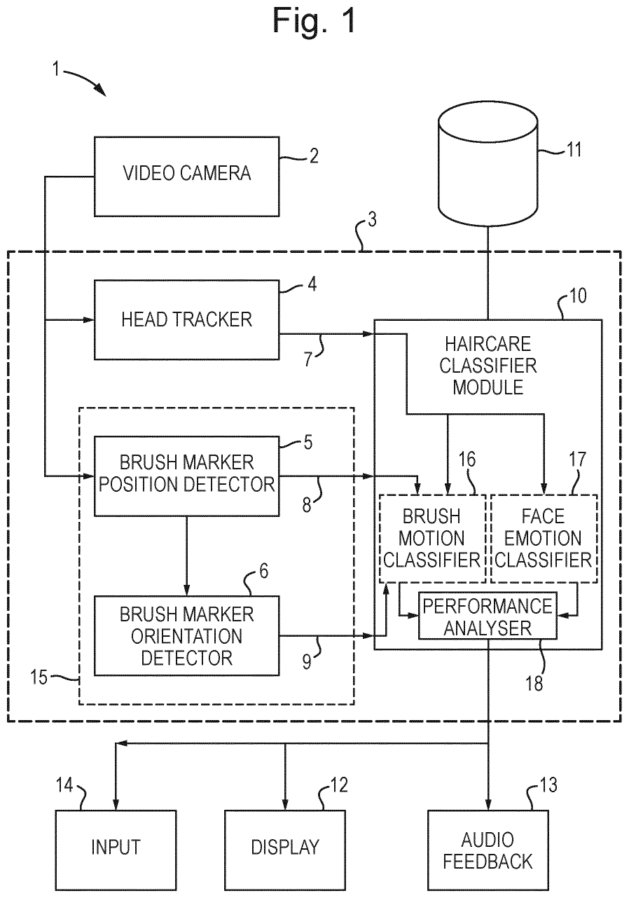

With reference to FIG. 1, a haircare monitoring system 1 for monitoring a user's haircare activity may comprise a video camera 2. The expression ‘video camera’ is intended to encompass any image-capturing device that is suitable for obtaining a succession of images of a user performing a haircare session. In one arrangement, the video camera may be a camera as conventionally found within a smartphone or other computing device.

The video camera 2 is in communication with a data processing module 3. The data processing module 3 may, for example, be provided within a smartphone or other computing device, which may be suitably programmed or otherwise configured to implement the processing modules as described below. The data processing module 3 may include a head tracking module 4 configured to receive a succession of frames of the video and to determine various features or parameters of a user's head and face. For example, the head tracking module 4 may determine landmarks on a user's face or head and an orientation of the user's face or head therefrom. As a further example, the head tracking module 4 may determine one or more facial action units corresponding to a facial muscle action. As a yet further example, the head tracking module 4 may classify a style of a user's hair.

The data processing module 3 may optionally include a brush tracking module 15 configured to receive a succession of frames of the video and determine position and motion parameters of a haircare implement used by the user in performing the haircare session. The haircare implement may be a hairbrush or hair straightener device, for example, and the haircare session may be a hair brushing or hair straightening session, for example. It will be appreciated that examples described with reference to a ‘brush’ (which is used synonymously with hairbrush herein) in the specific embodiments discussed below may also apply equally to other types of haircare implement instead of brushes.

The brush tracking module 15 may include a brush marker position detecting module 5 and a brush marker orientation estimating module 6. The position detecting module 5 may be configured to receive a succession of frames of the video and to determine a position of a brush within each frame. The brush marker orientation estimating module 6 may be configured to receive a succession of frames of the video and to determine/estimate an orientation of the brush within each frame.

The expression ‘a succession of frames’ is intended to encompass a generally chronological sequence of frames, which may or may not constitute each and every frame captured by the video camera and is intended to encompass periodically sampled frames and/or a succession of aggregated or averaged frames.

The respective outputs 7, 8, 9 of the head tracking module 4, the brush marker position detecting module 5 and the brush marker orientation detecting module 6 may be provided as inputs to a haircare classifier 10. The haircare classifier 10 is configured to determine haircare events and/or haircare performance parameters of the haircare session. In examples comprising a brush tracking module 15, the haircare classifier 10 can comprise a brush motion classifier 16 configured to determine one or more brushing parameters. The brushing parameters may include mechanical parameters such as position and linear and/or angular speed, velocity and acceleration. Linear motions may be made with reference to the frame of the image (camera) or with reference to the 3D object itself.

Rotational features are with reference to the 3D object itself. The brushing parameters may also include a brush path or trajectory corresponding to a particular brush stroke. In some examples, the haircare classifier 10 may comprise a face emotion classifier 17 configured to determine one or more emotional expressions of the user such as pain, frustration, confusion or happiness.

The head tracking module 4 can include a haircare performance analyser 18 which can receive brushing parameters from the brush motion classifier 16 and/or receive the one or more emotional expressions from the face emotion classifier 17. As discussed further below under section 4C, the performance analyser 18 may process the brushing parameters and/or emotional expressions to analyse the haircare performance of the user. The performance analyser 18 may analyse the haircare performance by detecting one or more haircare events or one or more haircare performance parameters. A haircare event may include any of: a detangling event, an inadequate brush stroke, an inadequate brush-hair contact and the like. Performance parameters may include any of a hairbrush applied force, a hairbrush-hair grip, a user satisfaction or the order in which the user carries out the components that make up the grooming task. In one example, the classifier 10 is configured to be able to classify each video frame of a brushing action of the user.

A suitable storage device 11 may be provided for programs and haircare data. The storage device 11 may comprise the internal memory of, for example, a smartphone or other computing device, and/or may comprise remote storage. A suitable display 12 may provide the user with, for example, visual feedback on the real-time progress of a haircare session and/or reports on the efficacy of current and historical haircare sessions.

A performance parameter for a user could change or improve between sessions. As such, analysing the performance of the user performing haircare may involve determining a performance parameter based data obtained in a current haircare session and data obtained in one or more previous haircare sessions.

A further output device 13, such as a speaker, may provide the user with audio feedback. The audio feedback may include real-time spoken instructions on the ongoing conduct of a haircare session, such as instructions on when to move to another head region or guidance on hair brushing action. An input device 14 may be provided for the user to enter data or commands. The display 12, output device 13 and input device 14 may be provided, for example, by the integrated touchscreen and audio output of a smartphone.

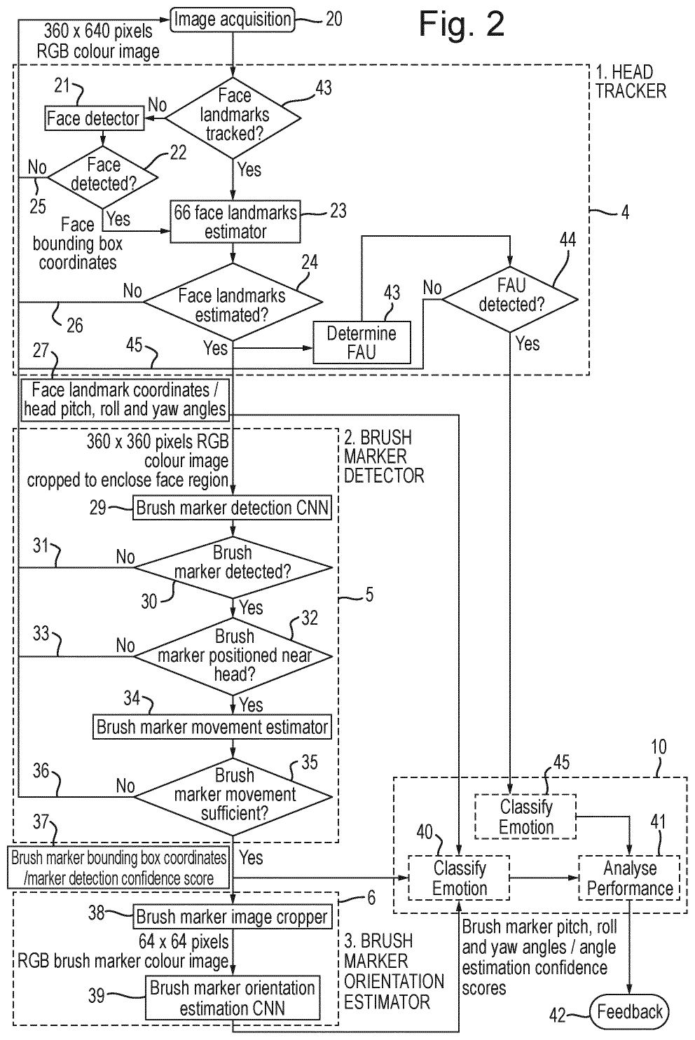

The functions of the various modules 4-6 and 10 above will now be described with reference to FIG. 2.

### 1. Head Tracking Module

The head tracking module 4 may receive (box 20) as input each successive frame or selected frames from the video camera 2. In one arrangement, the head tracking module 4 takes a 360×640-pixel RGB colour image, and attempts to detect the face (or head) therein (box 21). If a face is detected (box 22) the face tracking module 4 estimates the X-Y coordinates of a plurality of face landmarks (or more generally head landmarks) therein (box 23). The resolution and type of image may be varied and selected according to requirements of the imaging processing.

In one example, up to 66 face landmarks may be detected, including edge or other features of the head, nose, eyes, cheeks, ears and chin. Preferably the landmarks include at least two landmarks associated with the user's nose, and preferably at least one or more landmarks selected from head feature positions (e.g. corners of the head, centre of the head) and eye feature positions (e.g. corners of the eyes, centres of the eyes). The head tracking module 4 also preferably uses the landmarks to estimate some or all of head pitch, roll and yaw angles (box 27). The head tracking module 4 can also use the face landmarks to determine one or more facial action units (FAUs) (box 43). FAUs form part of the facial action coding system (FACS) known in the art. In some examples, the head tracker module 4 may determine other FACS parameters such as facial action descriptors (FADs). The head tracking module 4 may deploy conventional face tracking techniques such as those described in E. Sánchez-Lozano et al. (2016). “Cascaded Regression with Sparsified Feature Covariance Matrix for Facial Landmark Detection”, Pattern Recognition Letters.

If the head tracking module 4 fails to detect a face (box 22), the module 4 may be configured to loop back (path 25) to obtain the next input frame and/or deliver an appropriate error message. If the landmarks are not detected, or insufficient numbers of them are detected (box 24), the head tracking module 4 may loop back (path 26) to acquire the next frame for processing and/or deliver an error message. If FAUs are not detected (box 44), the head tracking module 4 may also loop back (path 45) in a similar manner. Where face detection has been achieved in a previous frame, defining a search window for estimating landmarks, and the landmarks can be tracked (e.g. their positions accurately predicted) in a subsequent frame (box 43) then the face detection procedure (boxes 21, 22) may be omitted.

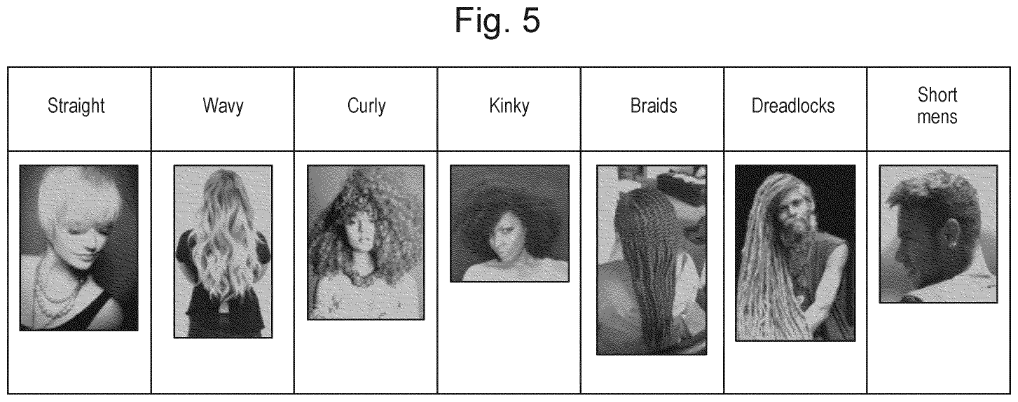

In some examples, the head tracking module 4 may determine a hair style or hair type of the user. FIG. 5 illustrates example hair styles including straight, wavy, curly, kinky, braids, dreadlocks and short men's. The head tracking module 4 may make such a determination on images prior to and following a haircare session. For example a user may record a “selfie” image at the beginning and end of a session. The head tracking module 4 may perform segmentation on an image to isolate pixels relating to the user's hair. The head tracker module 4 may implement a convolutional neural network (CNN) and may have been trained on a dataset composed of labelled hair-style images from various users with various head orientations and lighting conditions taken from brushing videos collected for training purposes. The head tracker module 4 may output a classified hair type to the haircare classifier 10. It is noted that in any one frame, different regions of the hair style may be given different classes (i.e. some parts maybe straight, some parts wavy) or one overall class depending on what is relevant. If the head tracking module 4 cannot determine a hair type, the head tracking module 4 may loop back to acquire the next image for processing.

In one example, a Face Detection Facial Point tracking (FACS) and expressed emotion recognition module may be configured to:
Step 1) Face detection—drawing a bounding box around the face and extracting the face patch from the image captured by the camera.
Step 2) Facial point tracking—64 Facial points are fitted to the face patch and these points are updated each frame until the face is lost.
Step 3) Estimation of Facial Action Unit System (FACS) activation from facial points and visual appearance. Step 3 relies on steps 1 and 2.
Step 4) Pain estimation—the FACS activation is used to estimate the expressed pain <-based on Solomon's pain intensity (PSPI) scale we modify it to make it more robust. Step 4 relies on Step 3.

### 2. Brush Marker Position Detecting Module

The brush used may be provided with brush marker features that are recognizable by the brush marker position detecting module 5. The brush marker feature acts as a fiducial marker. The brush marker features may, for example, be well-defined shapes and/or colour patterns on a part of the brush that will ordinarily remain exposed to view during a haircare session. The brush marker features may form an integral part of the brush, or may be applied to the brush at a time of manufacture or by a user after purchase for example.

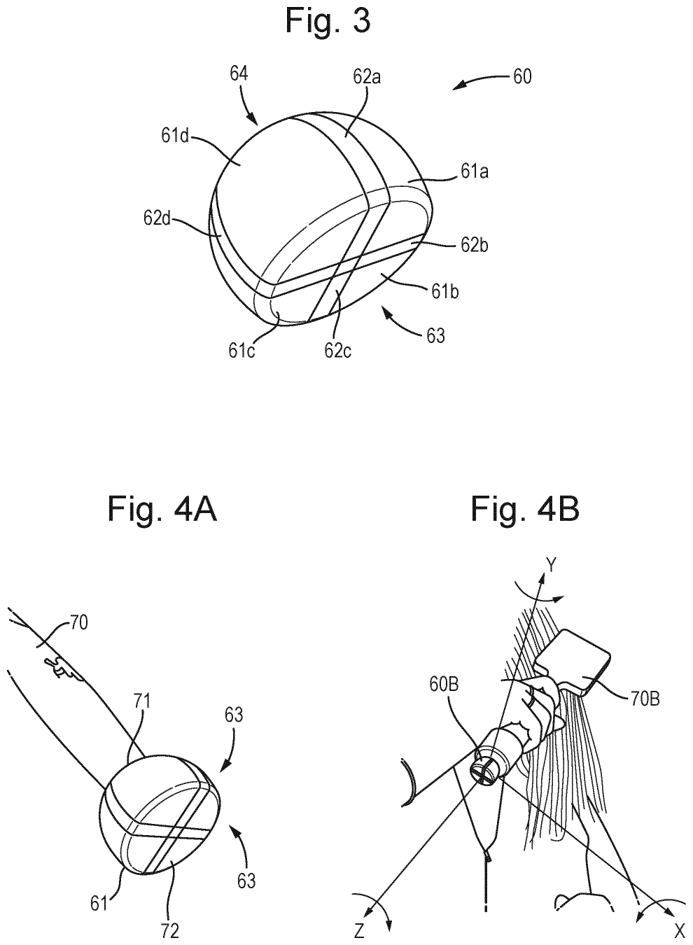

One particularly beneficial approach is to provide a structure at an end of the handle of a haircare implement, such as a hairbrush, i.e. the opposite end to the bristles. The structure can form an integral part of the brush handle or can be applied as an attachment or ‘dongle’ after manufacture. A form of structure found to be particularly successful is a generally spherical marker 60 (FIG. 3) having a plurality of coloured quadrants 61a, 61b, 61c, 61d disposed around a longitudinal axis (corresponding to the longitudinal axis of the brush). In some arrangements as seen in FIG. 3, each of the quadrants 61a, 61b, 61c, 61d is separated from an adjacent quadrant by a band 62a, 62b, 62c, 62d of strongly contrasting colour. The generally spherical marker may have a flattened end 63 distal to a handle receiving end 64, the flattened end 63 defining a planar surface so that the brush can be stood upright on the flattened end 63.

This combination of features has been found to be advantageous for both detecting the haircare implement in a typical grooming environment and determining its 3D orientation. The different colours enhance the performance of the structure and are preferably chosen to have high colour saturation values for easy segmentation in poor and/or uneven lighting conditions. The choice of colours can be optimised for the particular model of video camera in use. For consumer facing applications, the choice of colours may be such that they function well with a range of consumer image sensors on user devices. As seen in FIG. 4A, the marker 60 may be considered as having a first pole 71 attached to the end of a brush handle 70 and a second pole 72 in the centre of the flattened end 63. The quadrants 61 may each provide a uniform colour or colour pattern that extends uninterrupted from the first pole 71 to the second pole 72, which colour or colour pattern strongly distinguishes from at least the adjacent quadrants, and preferably strongly distinguishes from all the other quadrants. In this arrangement, there may be no equatorial colour-change boundary between the poles. As also seen in FIG. 4A, an axis of the marker extending between the first and second poles 71, 72 is preferably substantially in alignment with the axis of the haircare implement/brush handle 70.

FIG. 4B illustrates a marker 60B attached to a hairbrush 70B. Various axes X, Y, Z of rotation of the brush are illustrated in FIG. 4B. Orientational motions may be determined in the 3D frame of the marker (and thus brush) using the labelled axes. Linear motions of the marker 60B may be determined as 2D motions in the frame of the camera sensor.

The choice of contrasting colours for each of the segments may be made to optimally contrast with skin tones or hair colour of a user using the brush. In an example, red, blue, yellow and green are used. The colours and colour region dimensions may also be optimised for the video camera 2 imaging device used, e.g. for smartphone imaging devices. The colour optimisation may take account of both the imaging sensor characteristics and the processing software characteristics and limitations.

Modifications to the design of brush marker features are possible. Any design in principle could be used, providing that there can be made a 1-2-1 correspondence between the visual appearance of the marker in a frame of the video, at the video resolution, and the orientation of the marker, at least for the orientation of interest and there is sufficient annotatable training data to train a ML model to learn the orientations. In preferred examples, the diameter of the marker 60 is between 25 and 35 mm (and in one specific example approximately 28 mm) and the widths of the bands 62 may lie between 2 mm and 5 mm (and in the specific example 3 mm).

In one arrangement, the brush marker position detecting module 5 receives face position coordinates from the head tracking module 4. The resulting image is then used by a CNN (box 29) in the brush marker detecting module 5, which returns a list of bounding box coordinates of candidate brush marker detections each accompanied with a detection score, e.g. ranging from 0 to 1.

The detection score indicates confidence that a particular bounding box encloses the brush marker. In one arrangement, the system may provide that the bounding box with the highest returned confidence corresponds with the correct position of the marker within the image provided that the detection confidence is higher than a pre-defined threshold (box 30). If the highest returned detection confidence is less than the pre-defined threshold, the system may determine that the brush marker is not visible. In this case, the system may skip the current frame and loop back to the next frame (path 31) and/or deliver an appropriate error message. In a general aspect, the brush marker position detecting module exemplifies a means for identifying, in each of a plurality of frames of the video images, predetermined marker features of a brush in use from which a brush position and orientation can be established.

If the haircare implement marker is detected (box 30), the haircare implement marker detecting module 5 checks the distance between the face (or head) landmarks and the haircare implement marker coordinates (box 32). Should these be found too far apart from one another, the system may skip the current frame and loop back to the next frame (path 33) and/or return an appropriate error message. The brush-to-head distance tested in box 32 may be a distance normalised by nose length, as discussed further below.

The system may also keep track of the haircare implement marker coordinates over time, estimating a marker movement value (box 34), for the purpose of detecting when someone is not using the haircare implement. If this value goes below pre-defined threshold (box 35), the brush marker detecting module 5 may skip the current frame, loop back to the next frame (path 36) and/or return an appropriate error message.

The brush marker detecting module 5 is preferably trained on a dataset composed of labelled real-life brush marker images in various orientations and lighting conditions taken from brushing videos collected for training purposes, which may be extended using data augmentation techniques typical in machine learning. Every image in the training dataset can be annotated with the brush marker coordinates in a semi-automatic way. The brush marker detector may be based on an existing pre-trained object detection convolutional neural network, which can be retrained to detect the brush marker. This can be achieved by tuning an object detection network using the brush marker dataset images, a technology known as transfer learning.

### 3. Brush Marker Orientation Estimator

The brush marker coordinates, or the brush marker bounding box coordinates (box 37), are passed to the brush orientation detecting module 6 which may crop the brush marker image and resize it (box 38) to a pixel count which may be optimised for the operation of a neural network in the brush marker orientation detecting module 6. In an example, the image is cropped/resized down to 64×64 pixels. The resulting brush marker image is then passed to a brush marker orientation estimator convolutional artificial neural network (CNN—box 39), which returns a set of pitch, roll and yaw angles for the brush marker image. Similar to the brush marker position detection CNN, the brush marker orientation estimation CNN may also output a confidence level for every estimated angle ranging from 0 to 1.

The brush marker orientation estimation CNN may be trained on any suitable dataset of images of the marker under a wide range of possible orientation and background variations. Every image in the dataset may be accompanied by the corresponding marker pitch, roll and yaw angles.

In some implementations, the brush marker position detector 5 and the brush orientation detecting module 6 may be provided by the same functional unit in hardware and/or software.

### 4. Haircare Classifier

### 4A. Brush Motion Classifier

The brush motion classifier 16 accumulates the data generated by the three modules described above (face tracking module 4, brush marker position detecting module 5, and brush marker orientation detection module 6) to extract a set of features designed specifically for the task of haircare implement classification (box 40).

Facial landmark coordinates (such as eyes, nose and mouth positions) and brush coordinates are preferably not directly fed into the classifier 10 but used to compute various relative distances and angles of the brush with respect to the face, among other features as indicated above. In this way, the brush motion classifier 16 can determine a relative brush position and relative brush orientation relative to the user's head poise.

The brushing motion classifier may output one or more brushing parameters. The brushing parameters may include mechanical parameters including any of: absolute and/or relative brush position and orientation; linear velocity; angular velocity; linear acceleration; and angular acceleration. The brush motion classifier may determine dynamic parameters (velocity, acceleration etc) based on changes in position and orientation between successive frames. The mechanical parameters may comprise an absolute magnitude or values along one or more axes. The brushing parameters may also include brush stroke parameters relating to individual brush strokes, such as brush path or trajectory encompassing the plane and curvature of the brush stroke. The brushing parameters may also include more general parameters such as a brushed region of the hair (front, middle, back for each of right side and left side of head).

The brush length is a projected length, meaning that it changes as a function of the distance from the camera and the angle with respect to the camera. The head angles help the classifier take account of the variable angle, and the nose length normalisation of brush length helps accommodate the variability in projected brush length caused by the distance from the camera.

The brush motion classifier 16 may be trained on a dataset of labelled videos capturing person's brushing. The dataset may be captured of a brush with a marker comprising a calibrated accelerometer such that the labelling can include the mechanical parameters. Every frame in the dataset may be labelled with brushing parameters from the accelerometer and/or by an action the frame depicts. The actions may include “IDLE” (no brushing), “MARKER NOT VISIBLE”, “OTHER” and brushing actions. In this way, the classifier can be trained to understand the relationship between the position, orientation and presence of the brush marker and the brushing parameters.

### 4B. Face Emotion Classifier

The face emotion classifier 17 can receive FAUs from the head tracker 4 and determine one or more facial expressions based on the FAUs (box 45). A value of an FAU may be an intensity of the associated facial muscle movement. In some examples, the face emotion classifier 17 can determine a score based on a set of FAUs. The face emotion classifier 17 may normalise FAU values prior to determining a score based on a normalization procedure, which can minimise a subject-specific AU output variation. The normalization procedure may be based on user data captured during a calibration routine.

As outlined above, the haircare routine may be associated with one or more painful experiences resulting from detangling and associated tugging on the scalp. The face emotion classifier 17 can determine a pain expression of the user based on the FAUs. In some examples, the face emotion classifier can determine a pain expression based on the Prkachin and Solomon Pain Intensity Metric (PSPI) scale. The PSPI scale can be calculated based on:

$${Pain}_{PSPI} = Intensity ( AU 4 ) + Max ( Intensity ( AU 6 , AU 7 ) ) + Max ( Intensity ( AU 9 , AU 10 ) ) + Intensity ( AU 43 )$$

Where AU4 is the FAU “Eye-brow lowerer”; AU6 is the FAU “cheek raiser”; AU7 is the FAU “eye-lid tightener”; AU9 is the FAU “nose wrinkle”; AU10 is the FAU “upper lip raiser”; and AU43 is the FAU “eye closed.” In some examples, a pain score may be determined according to the equation but without the inclusion of the AU4 and AU9 dependence. In some examples, the face emotion classifier 17 may filter images prior to calculating a pain score. For example, the face emotion classifier 17 may remove images with a large out of plane head rotation; remove images in which no brushing is occurring or no brush is present; remove images in which hair occludes a significant portion of the face; and remove images with a low face detection confidence. The face emotion classifier 17 may receive any of the outputs 7, 8, 9 from the head tracking module 4 or brush tracking module 15 for performing the filtering process.

Study data comprising smart phone image sequences of user's hair brushing was processed according to the above equation. A significant increase in PSPI score was identified for 75 to 80% of images in which the user exhibited a pain expression (assessed manually). Further analysis revealed that false positives (a high pain score with no associated pain expression) could be reduced by removing the AU4 and AU9 dependencies above. AU4 and AU9 were found to me more error prone and typically correlated with other indications of pain. Therefore, false positives could be reduced without affecting accuracy. Other methods for reducing false positives included: filtering images with a large out of plane head rotation; filtering of images in which no brushing is occurring or no brush is present; filtering images in which hair occludes a significant portion of the face; and ignoring images with a low face detection confidence.

In some examples, the face emotion classifier 17 may determine a happiness expression of the user based on the FAUs. In some examples, the face emotion classifier can determine a happiness expression based on AU6 (“cheek raiser”) and AU12 (“lip corner puller”) intensities. As AU6 can also be present in a pain expression, the face emotion classifier 17 may also determine a happiness expression by reducing a happiness score based on the presence of other pain indicators (AU4, AU10, AU43). As an example, the face emotion classifier 17 may determine a happiness score as:

$$Happiness = Max [ \{ 0.5 \times ( AU 6 + AU 12 ) - Max ( AU 4 , AU 10 , AU 43 ) \} , 0 ]$$

In some examples, the face emotion classifier 17 may filter images prior to calculating a happiness score. For example, the face emotion classifier 17 may remove images with a large out of plane head rotation; remove images in which hair occludes a significant portion of the face; and remove images with a low face detection confidence. The face emotion classifier 17 may receive any of the outputs 7, 8, 9 from the head tracking module 4 or brush tracking module 15 for performing the filtering process.

Study data comprising smart phone image sequences of user's hair brushing was processed according to the above equation. A significant increase in happiness score was identified for 80% of images in which the user exhibited a happiness expression (assessed manually).

### 4C. Haircare Performance Analyser

The haircare performance analyser 18 can receive brushing parameters from the brush motion classifier 16 and/or receive the one or more emotional expressions from the face emotion classifier 17. The performance analyser 18 may process the brushing parameters and/or emotional expressions to analyse the haircare performance of the user (box 41). The performance analyser 18 may analyse the haircare performance by detecting one or more haircare events or one or more haircare performance parameters. A haircare event may include any of a detangling event, an inadequate brush stroke, an inadequate brush-hair contact and the like. Performance parameters may include any of a hairbrush applied force, a brush-hair grip or a user satisfaction or any proxy measure for any of these parameters when suitably calibrated. The performance analyser may comprise one or more performance models related to the haircare performance. Example performance models include: a detangling model for detecting detangling events; a force model for determining a force applied to a hairbrush (or a representative (proxy) of applied force); and a satisfaction model for determining a user's satisfaction with the haircare session. A performance model may comprise a machine learning algorithm trained on manually labelled data. The performance analyser 18 can advantageously assess the performance of a user's haircare routine and an associated health of the user's hair. The system 1 can provide feedback to the user to improve the haircare routine and the associated health of the user's hair.

### Detangling Model—Detecting Detangling Events

As outlined above the detangling process during the haircare grooming process can be a painful and unpleasurable experience for the user. The performance analyser 18 can use a detangling model to detect a detangling event based on outputs from the face emotion classifier 17 and/or the brush motion classifier 18. As discussed below, feedback may then be provided to the user for mitigating or preventing future detangling events.

In some examples, the performance analyser 18 may detect a detangling event based on an indication of pain from the face emotion classifier 17 combined with a substantially zero velocity or acceleration from the brush motion classifier 16.

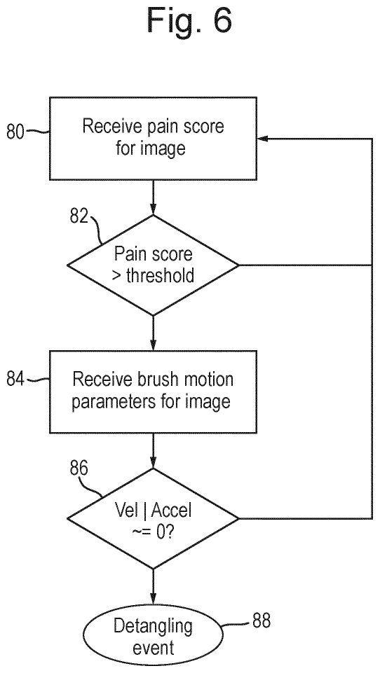

FIG. 6 illustrates a method of detecting a detangling event as may be performed by the performance analyser 18. At a first step 80, the performance analyser 18 receives a pain score for the image (or sequence of images) being processed from the face emotion classifier 17. At a second step 82, the performance analyser 18 compares the pain score with a detangling pain threshold. If the pain score is less than the detangling pain threshold the process loops back to the first step 80 to receive pain data from subsequent images. If the pain score is greater than or equal to the detangling pain threshold, the process proceeds to third step 84. In some examples, the performance analyser 18 may compare the pain score to the detangling pain threshold for a single image. In other examples, the performance analyser 18 may compare the pain score to the detangling pain threshold for a plurality of images, corresponding to a longer duration. The second step 82 may require that the pain score exceeds the detangling pain threshold for each of the plurality of images, or that an average pain score exceeds the threshold, to proceed to the third step 84. In this way, a more robust detection of a detangling pain event can be detected.

At the third step 86, the performance analyser 18 receives brush motion parameters from the brush motion classifier 16. The performance analyser 18 may compare one or more brush motion parameters against corresponding detangling thresholds to detect detangling motion. As illustrated, the performance analyser 18 may determine if one or more of: linear or angular velocity; and linear or angular acceleration are substantially equal to zero. In other words, if the brushing parameter is less than a corresponding detangling motion threshold. If the one or more brushing parameters are greater than their corresponding detangling motion threshold, the process loops back to the first step 80. If (each or any of) the one or more brushing parameters is less than their corresponding detangling motion threshold, the performance analyser 18 proceeds to step 88 and outputs an identified detangling event. The linear or angular velocity/acceleration may comprise a linear or angular velocity/acceleration along or about one or more axes (as discussed previously in relation to FIG. 4B) or may relate to an absolute magnitude of linear or angular velocity/acceleration.

In the example of FIG. 6, the performance analyser 18 can identify a painful detangling event by jointly analysing the pain signal for pain above a threshold and the velocity or acceleration signal from the motion parameters for low or zero velocity/acceleration. In other examples, the performance analyser 18 may omit steps 80 and 82 or 84 and 86 and detect a detangling event based either on the pain data from the face emotion analyser 17 or on the brush motion data from the brush motion analyser 16.

Determining a detangling event based on an acceleration or velocity being substantially zero reduces calibration requirements for the system 1 because the performance analyser 18 is only analysing a turning point in acceleration or velocity rather than a rate of change of the parameter before or after the turning point. Therefore, the detangling detection method can be advantageously independent of a variation in brush force across different users. In addition, the PSPI score provides comparable results for different users, further reducing calibration requirements.

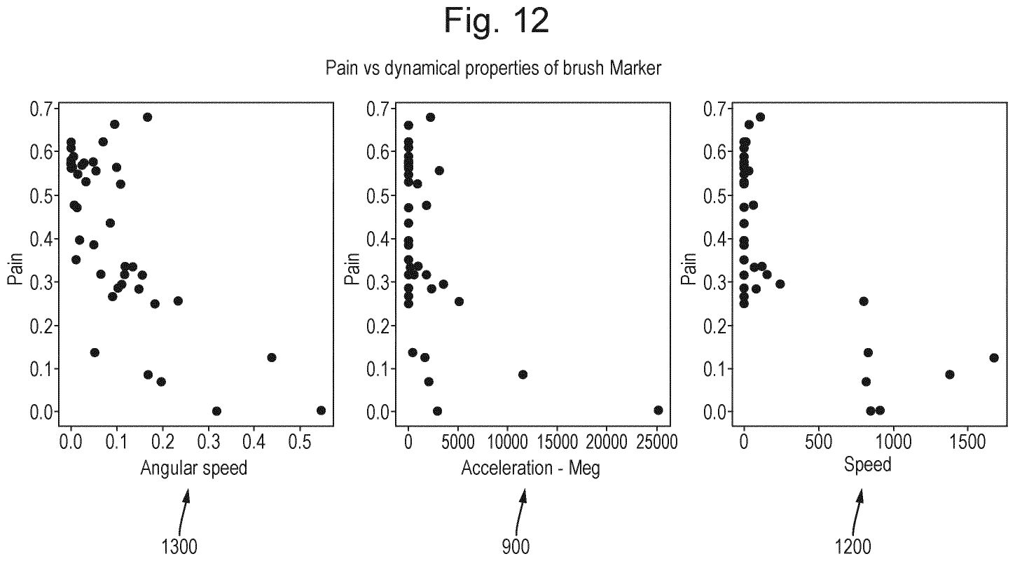

In some examples, during step 86 the performance analyser 18 may analyse one or more other brush motion parameters. The performance analyser 18 may analyse one or more brush motion parameters to determine that the user is in the process of brushing their hair. For example, the performance analyser 18 may receive such an indication from the brush motion classifier 16 or from the brush marker position detecting module 5 (as described above in relation to FIG. 2). In some examples, the performance analyser 18 may analyse a trajectory of the brush or parameters relating to the trajectory to identify sudden deacceleration and/or jerking motion as the brush sticks in tangled hair. In some examples, the performance analyser 18 may compare the brush position to one or more predetermined positions known to be prone to tangles, as discussed further below in relation to FIG. 12.

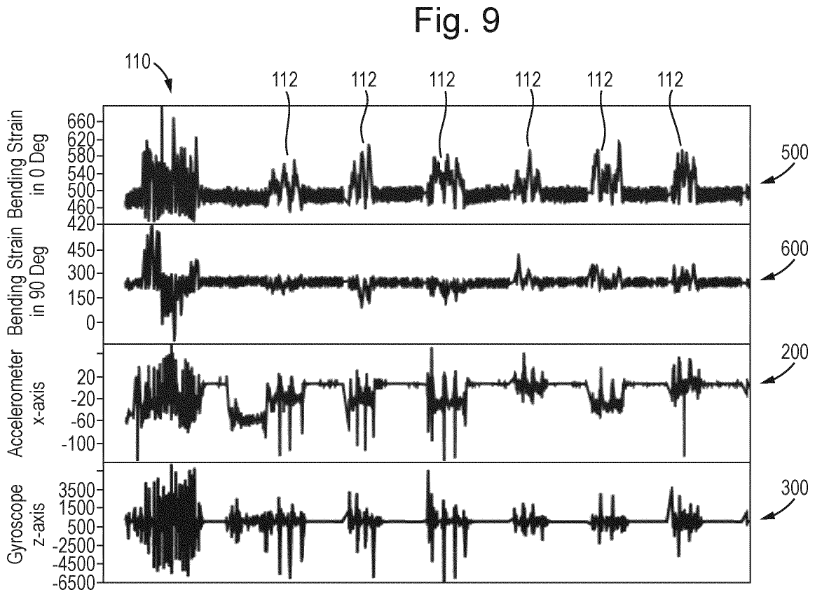

In some examples, as discussed below in relation to FIG. 9, the performance analyser 18 may comprise a machine learning (ML) algorithm, such as an artificial neural network, that has been trained using data comprising images of a user performing a haircare routine under known conditions. The images in the training data may be manually labelled to identify detangling events. The population of consumers used to build the ML model may be chosen to tailor the model to a particular demographic (e.g. population with a particular base hair type or perhaps an older (more frail/less dexterous) population group. The ML algorithm may then identify one or more brush motion parameters associated with detangling events. In step 86 of the process of FIG. 6, the performance analyser 18 may determine a detangling event based on one or more of these identified brush motion parameters exceeding a corresponding threshold.

In some examples, following identification of a detangling event (step 88) the performance analyser 18 or classifier module 10 may perform further analytics on one or more brush motion parameters in the period of time (and associated images) around the detangling event. In this way, the classifier may extract other brush motion parameters identifying the effectiveness of a product formulated for less tangles or less tight tangles. For example, the peak acceleration following a detangling event or a length of time that the brush remains stationary may provide quantitative insight on the effectiveness of the formulated product. In this way, the system 1 can track, monitor and compare the effectiveness of one or more products for a particular user.

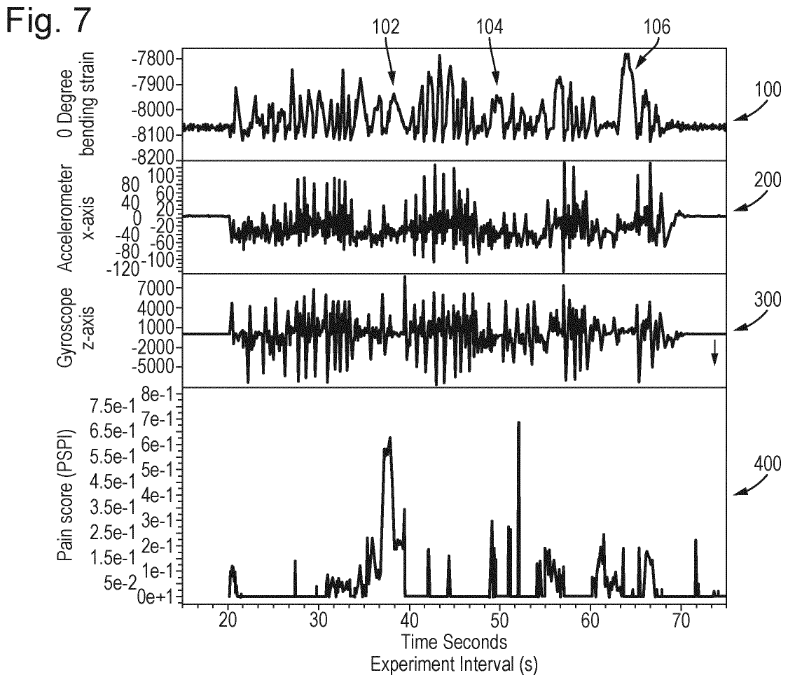

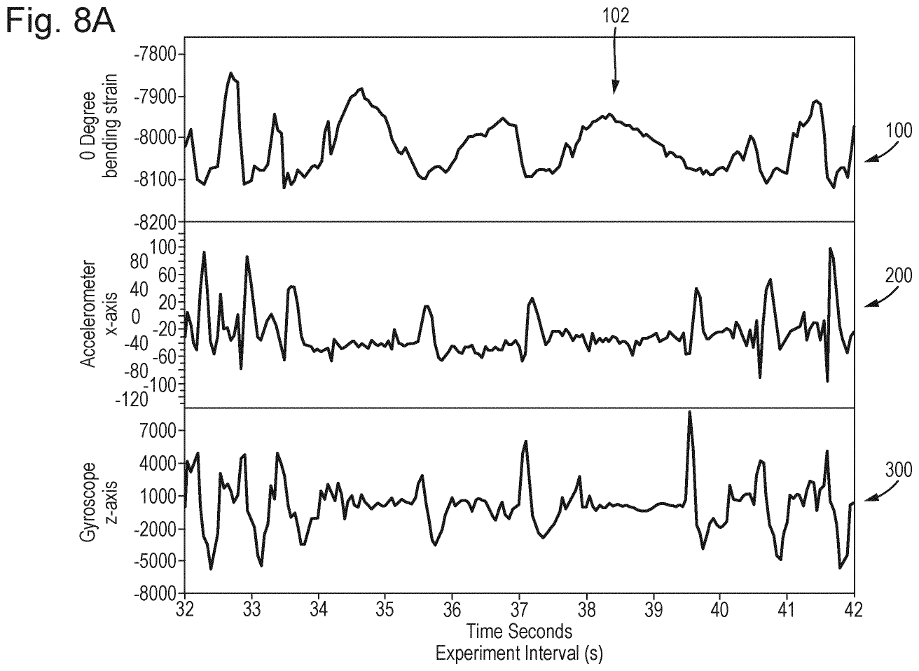

FIGS. 7 to 14 illustrate experimental data supporting the relationships underpinning detangling event detection based on pain and/or brush motion.

FIG. 7 illustrates study data collected from a haircare session monitored via a smartphone camera. The camera captured a sequence of images of a user brushing their hair with a force brush. The force brush included a plurality of sensors including a force sensor, an accelerometer and a gyroscope for measuring a plurality of mechanical parameters associated with the brush movement. The figure includes plots of lateral detangling force as represented by 0 degree strain data 100, accelerometer X axis data 200, and gyroscope Z axis data 300, all acquired from the force brush. All data 100, 200, 300, 400 are plotted against time.

The data 100, 200, 300 is annotated with qualitative descriptions taken from a corresponding video dataset. The qualitative descriptions include a first marker 102, a second marker 104 and a third marker 106. The three markers 102, 104, 106 each correspond to a detangling event. The first marker 102 provides an indicator of visible pain and tugging visible from the video data (assessed manually). The second marker 104 demonstrates visible tugging from the video data. The third marker 106 demonstrates visible pain from the video data. The figure further includes a plot of pain data 400 as determined by PSPI score from the corresponding video images. The pain signal 400 contains peaks corresponding to the first, second and third markers 102, 104 and 106, indicating the correlation between apparent pain assessed manually and the PSPI score.

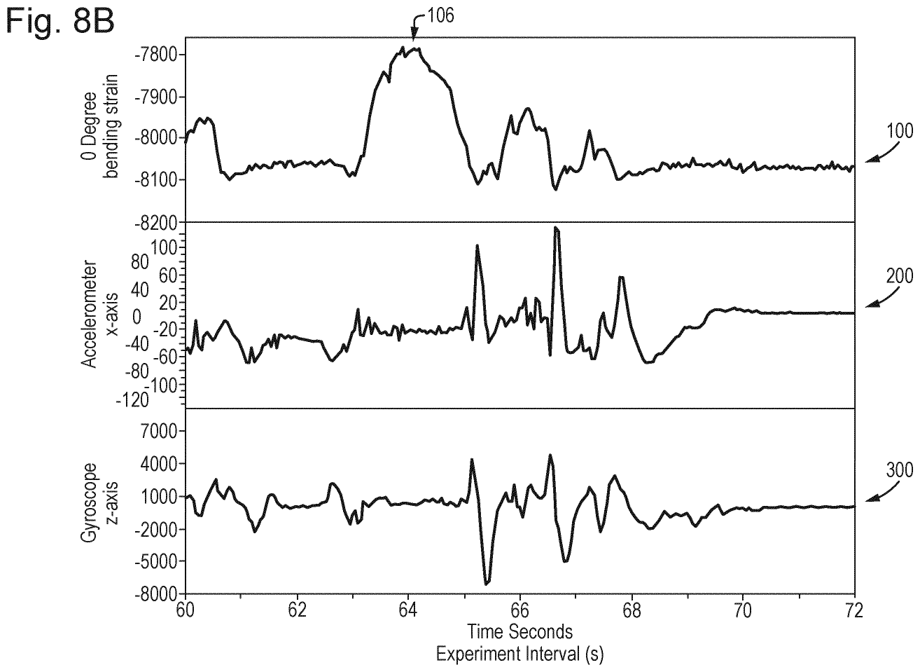

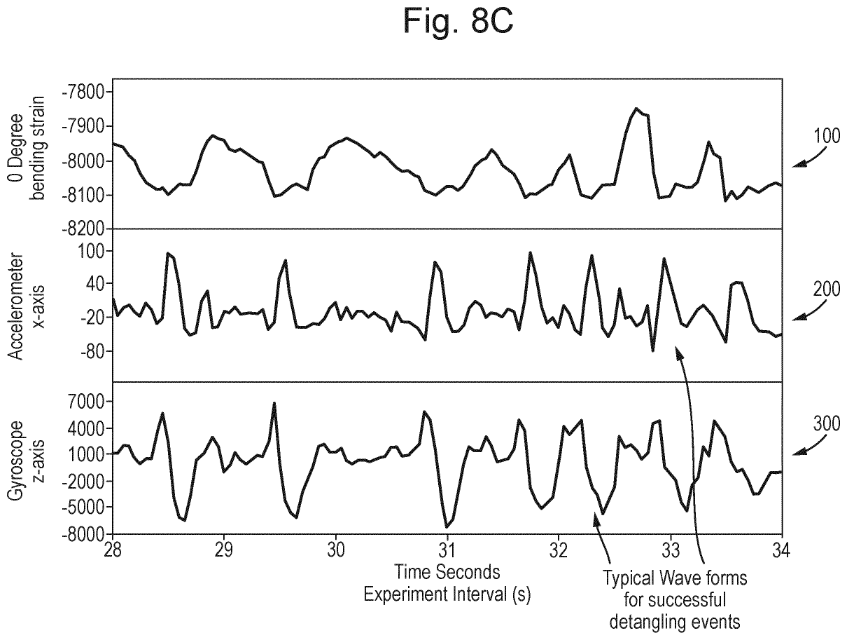

FIG. 8A illustrates a blown up portion of the profiles illustrated previously with respect to FIG. 7 around the first marker 102. In this example, the detangling lateral force profile includes elongated periods of force associated with a pain and a tugging event. At the same time, the acceleration along the X axis tends to 0 (with scaling offset). Also, the angular speed around the Z axis (gyroscopic Z) also reduces to zero during this period. Qualitatively, the twisting of the brush and the acceleration of the brush tends to 0 during the detangling event.

FIG. 8B illustrates a blown up portion of the profiles illustrated previously with respect to FIG. 7 around the third marker 106. In this example, the detangling lateral force profile includes one large elongated period of force associated with visible pain in the image. At the same time, similar to the effect seen at the first marker 104, the acceleration along the X axis and the angular speed around the Z axis tend to 0.

The data illustrates that the presence of detangling at the markers 102, 104, 106 is associated with each of: a broadening of lateral force peaks (indicative of sustained force required for detangling); substantially zero x-axis acceleration; substantially zero z-axis angular velocity; and an increase in PSPI score.

To illustrate the relationship further, FIG. 8C illustrates an expanded portion of another section of the data described previously with reference to FIG. 7. In the portion between times of 28 and 34 seconds, the detangling lateral force 100, the acceleration in the X axis 200 and the angular speed around Z (gyroscopic Z) 300 profiles are indicative of a period in which the user is not visibly experiencing pain or tugging of their hair. In this section, the periodicity of the brushing action is visible from the accelerometer profile 200 and angular speed around Z (gyroscopic Z) profile 300, and has a periodicity of around one second. Similarly, the change in accelerometer×profile 200 and gyroscopic Z profile 300 during this period is greater during the periodic cycles seen previously in FIGS. 8A and 8B in which visible pain and/or tugging are visible. The profiles 100, 200, 300 of FIG. 8C may be considered to illustrate successful detangling events.

FIG. 9 illustrates further study data captured according to a specific training protocol. The data may be used to train a machine learning (ML) algorithm to determine one or more parameters, such as one or more brush motion parameters, associated with a detangling event or other performance analysis (such as determining applied force as discussed further below). The data may be used to train the classifier 10 including the face emotion classifier 17, the brush motion classifier 16 and the performance analyser 18.

The protocol comprises a grooming/detangling process 110 followed by six periodically spaced heat styling segments 112 including a combination of brushing and blow drying. The six segments correspond to the brushing of three different regions (front, middle and back of the head) on each side of the head. Between the heat styling segments only blow drying is performed with no brushing. Detangling data 110 relating to the detangling process can be used as a discrete data set for training the classifier 10/performance analyser 18 to detect detangling events. Styling data 112 relating to the six heat styling segments can be used as a second discrete data set for training the classifier/performance analyser 18 to detect inappropriate brush use, inappropriate hair grip and other suboptimum use of the implement during style.

In this way, using data acquired using multiple subjects in a range of training protocols, classifiers may be developed more generally for the component parts of any hair grooming event and then applied to decompose uncontrolled grooming events into manageable parts for feedback and recommendation.

In this example, data was captured with a force brush (as described in relation to FIG. 7) with a marker applied to an end of the handle (as described in relation to FIG. 3). The data captured with the force brush includes: 0 degree (lateral) bending strain data 500; 90 degree bending strain data 600; x-axis accelerometer data 200; and z-axis gyroscope data 300.

Image data was also captured to determine motion parameters from the marker (using the head tracking module 4, the brush marker position detecting module 5, the brush orientation detecting module 6 and brush motion classifier 16) and FAUs and associated facial expressions (using head tracking module 4 and face emotion classifier 17).

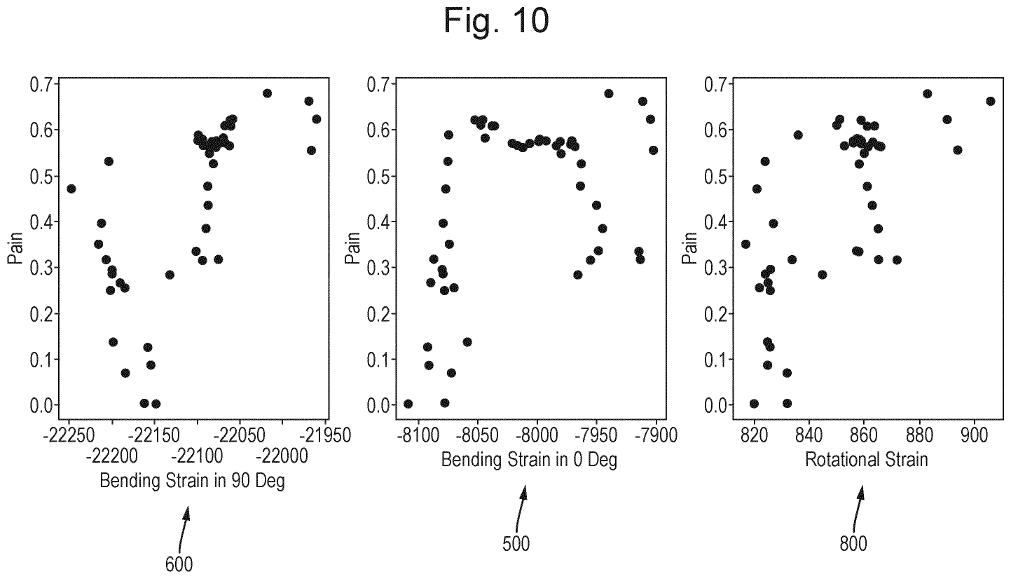

FIG. 10 shows plots of PSPI pain scores plotted against various force brush parameters for detangling process data 110 captured according to the protocol of FIG. 9. The plots include pain score against: (i) 90-degree bending strain 600; (ii) 0-degree bending strain 500; and (iii) rotational strain 800. The plots illustrate that higher rates of strains are generally associated with higher values of expressed pain. The strains may be considered to be “micro” linear and rotational deformations of the brush during use, so the brush gets stuck in a tangle and physically deforms a tiny amount (elastic but overtime time hysteresis), the history of these micro deformations over time depends both on the complexity of the tangle and the user's actions “to get out of the tangle”.

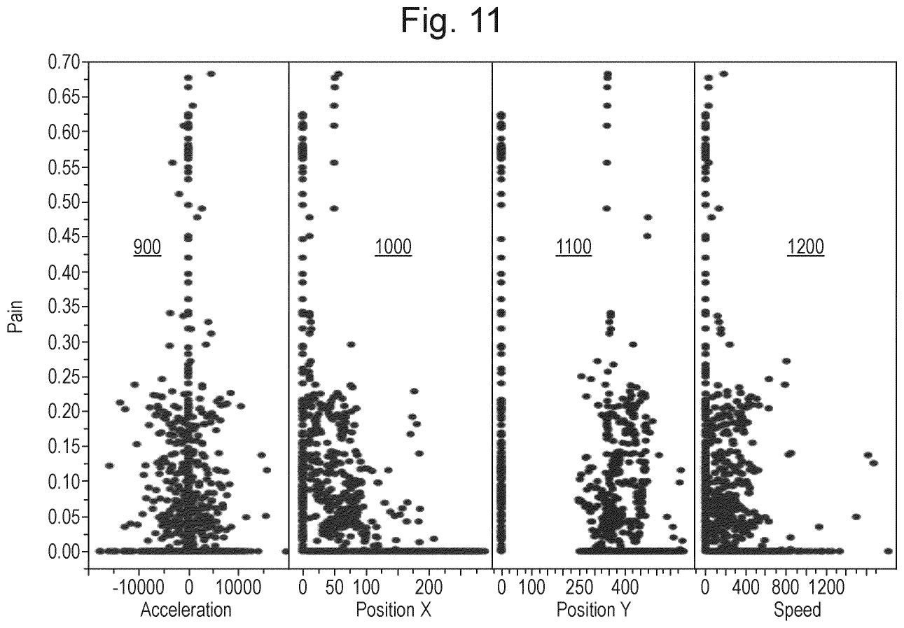

FIG. 11 shows plots of PSPI scores plotted against various motion parameters determined by the brush motion classifier based on the marker position and orientation in the image data. The plots include pain score against: (i) acceleration 900; x position 1000; y position 1100; and speed 1200. For the x and y position co-ordinates, a value of x=0, y=0 corresponds to the top left pixel of the image. The brush motion classifier 16 can determine the dynamic parameters—speed 1200 and acceleration 900—based on frame to frame changes in the positional values. In this example, the motion parameters default to zero if a value cannot be determined. In this example, pain values default to zero if a PSPI score cannot be determined.

The plots illustrate that high pain scores are associated with substantially zero speed and substantially zero acceleration, whereas lower pain scores are generally associated with a broader range of acceleration and velocity. Similarly, fixed values of x-position 1000 and y-position 1100 are associated with high pain scores, whereas lower pain scores are associated with a broad range of position co-ordinates.

FIG. 12 illustrates the data of FIG. 11 after filtering together with additional marker derived data for angular speed 1300. The filtering comprised: removing data for which pain score equals 0; removing data for which x position and y-position equals 0; for acceleration data, removing data for which acceleration was less than 22,000 pixels per second squared, and for speed data removing data for which speed is less than 800 pixels per second; and adding data back for which pain scores are greater than 0.25. The filtered data of FIG. 12 illustrates that higher pain scores are associated with the low speed and acceleration data points.

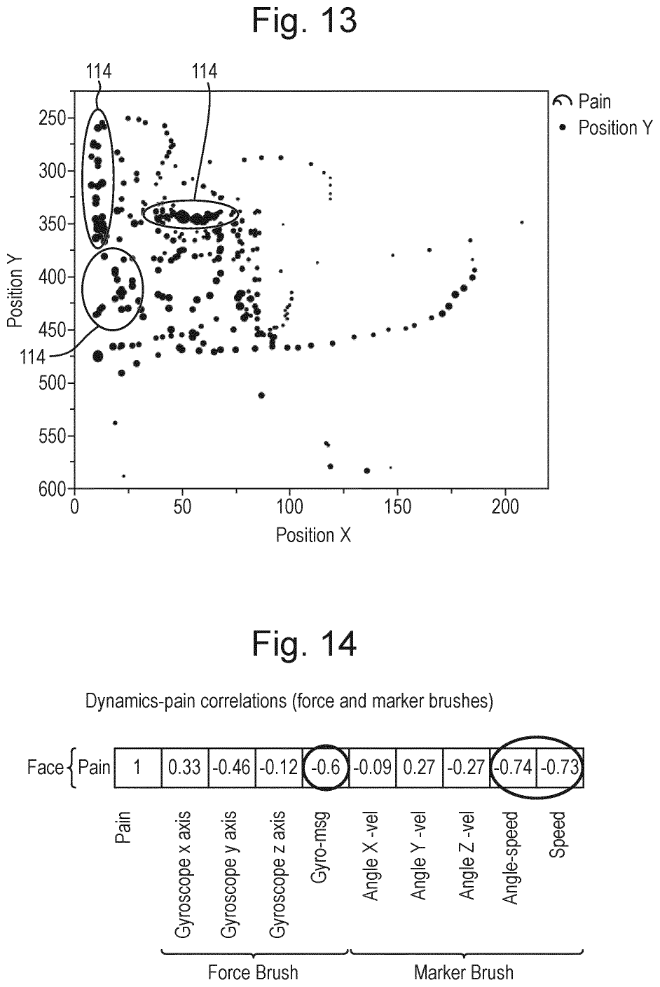

FIG. 13 illustrates the x-position 1000 plotted against the y-position 1100 of the data points of FIG. 11 with associated pain scores. In this example, the X and Y positions correspond with pixels of the camera and so relate to the camera frame of reference. The level of apparent pain is indicated by the size of the marker at a particular position. The data illustrates three regions 114 with a high concentration of high pain scores. In this way, the performance analyser 18 can identify regions where tugging and detangling events occur and the system 1 can provide feedback such as a recommendation of a spray/serum for easing tangles that could be applied to regions of pain 114.

FIG. 14 illustrates a correlation matrix identifying correlation strength between various parameters of the data underlying FIGS. 9 to 13 for the force brush (used for validation) and the marker brush. Strong correlations can be seen between: pain and absolute gyro values from the force brush; pain and angular speed, and pain and (linear) speed, both from the marker brush.

The data of FIGS. 7 to 14 illustrates that relationships exist between pain and brush motion and that both can be used to identify a detangling event. As a result the performance analyser 18 can determine a detangling event based on a pain score exceeding a detangling pain threshold and/or a linear or angular speed being less than a detangling speed threshold and/or an acceleration being less than a detangling acceleration threshold. As discussed below, following detection of a detangling event, the system 1 can provide user feedback to mitigate detangling pain and/or reduce the prevalence of future detangling events.

### Force Model—Determining Applied Brush Force

A long-standing challenge in haircare research is estimating the forces involved at different points of the styling process. Applied brush force to achieve a detangling event may directly depend upon formulation properties of the one or more haircare products used during the washing and haircare processes. A typical approach is to estimate applied brushing force is the use of the force brush (described above in relation to FIG. 7) however, such a brush is expensive and impractical to scale to a wide consumer base.

In one or more examples, the performance analyser 18 can use a force model to determine a force signal, representative of a relative level of force applied to the hairbrush, based on a pain score received from the face emotion classifier 17. As discussed below (and above in relation to FIGS. 7 to 8C), pain score is correlated with applied brush force and, as a result, the pain score can be used as a proxy for applied brush force. In this way, the disclosed systems 1 and methods are capable of reporting a proxy measure for force without requiring a force brush.

In some examples, pain score is only correlated with applied brush force during certain stages of a haircare session, for example at the start of a new brush stroke when the brush first grips the user's hair and may result in some tugging due to friction arising from the grip between the brush and the hair. For example, if the user is caught in a tangle then there may be only very constrained ways out the tangle, which will determine the force needed regardless of other factors and, at these points, the expressed pain is likely to be a good proxy for force. Therefore, in some examples, the performance analyser 18 may determine the force signal based on the pain score and brush motion parameters received from the brush motion classifier 16. The performance analyser 18 may determine the force signal based on the pain score when the brush motion parameters indicate the onset of a new brush stroke.

In a similar way to the detangling model, the force model may be a ML algorithm that can be trained on a data set similar to the one described in relation to FIGS. 7 to 14. That is data can be obtained for a user performing a haircare routine with a force brush with the marker of FIG. 3 attached. Data from the force brush itself and image data relating to a sequence of images captured during the haircare routine can be used to generate the model. The model may be trained based on force brush data and a corresponding PSPI score from the video image corresponding to the same time axis, such as that illustrated in FIG. 7 and FIG. 10. The model may further incorporate brush motion data derived by the brush motion classifier 16 from the brush marker position in the sequence of images, such as the data illustrated in FIGS. 11 and 12.

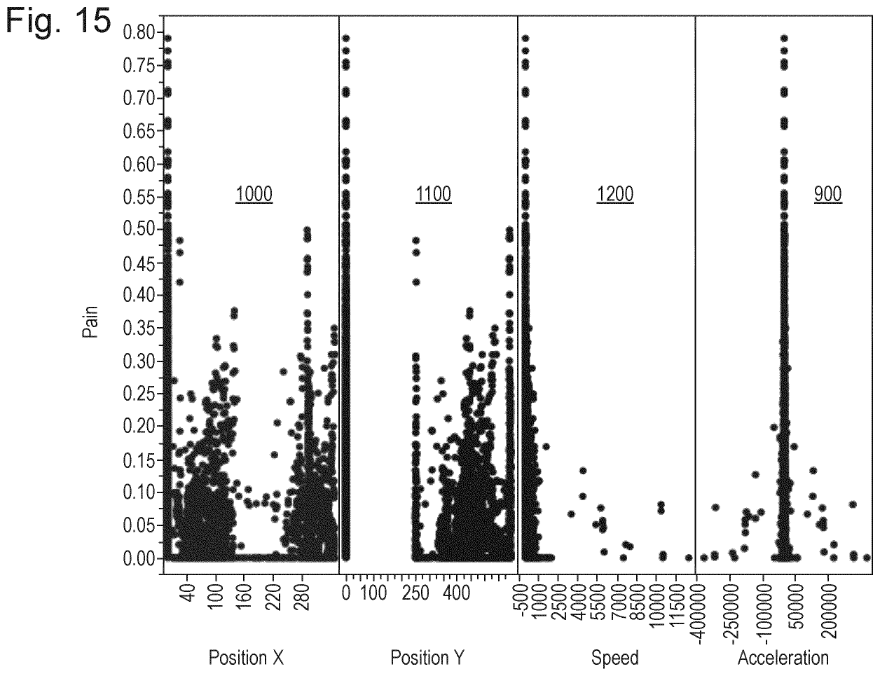

The training data may comprise the hair styling segments 112 of the training protocol data of FIG. 9. FIGS. 15 to 18 illustrate data for captured for styling segments 112 presented in a similar manner to the detangling process data of FIGS. 11 to 14. Such data can be useful for supplementing the force model with additional parameter dependence such as the onset of brush stroking as mentioned above.

FIG. 15 shows plots of PSPI scores plotted against various motion parameters in the same manner as FIG. 11 but for the hair styling segment data. The plots illustrate that high pain scores are associated with substantially zero speed and substantially zero acceleration, whereas lower pain scores are generally associated with a broader range of acceleration and velocity. Similarly, fixed values of x-position 1000 and y-position 1100 are associated with high pain scores, whereas lower pain scores are associated with a broad range of position co-ordinates.

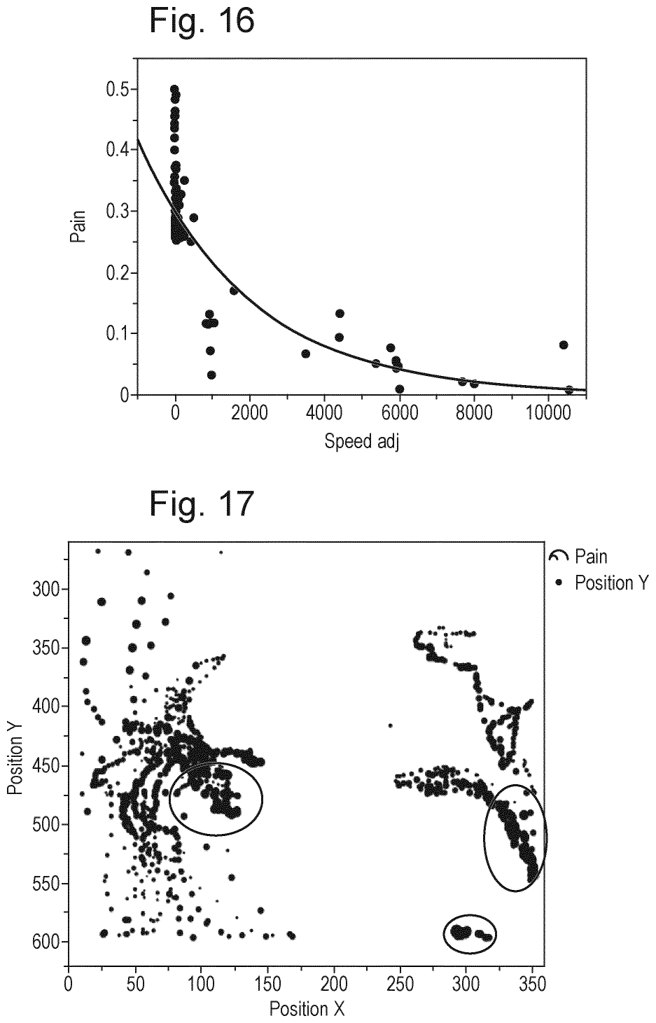

FIG. 16 illustrates the speed data of FIG. 15 after filtering. The filtering comprises: removing data for which pain score equals 0; removing data for which x position and y-position equals 0; removing data for which speed is less than 800 pixels per second; and adding data back for which pain scores are greater than 0.25. The filtered data of FIG. 15 illustrates a correlation between pain score and brush speed with a R2 fit of 0.75.

FIG. 17 illustrates the x-position 1000 plotted against the y-position 1100 of the data points of FIG. 15 with their associated pain scores. The data illustrates three regions 116 with a high concentration of high pain scores. In this way, the performance analyser 18 can identify regions where the applied brush force is too high. The system 1 can provide feedback such as a recommendation of a spray/serum for reducing friction between the brush and the hair that could be applied to regions of pain 116.

Using the force model, the performance analyser 18 can monitor the pain score and the proxy force signal from the captured video images. In this way, the system 1 can track, monitor and compare the effectiveness of one or more formulated products for a particular user based on the corresponding force signal (which may be an average force signal over a haircare session). Such product effectiveness monitoring may be particular advantageous during product development or consumer studies or for a particular end-user looking to compare different haircare products.

In some examples, the performance analyser may apply the force model in combination with the detangling model to further characterise a particular user and provide more personalised feedback. For example the performance analyser may divide users into four user types based on the number of detangling events and the level of force indicated by the force signal: “low force, lots of tangles”; “high force lots of tangles”; “low force, few tangles”; and “high force few tangles.”

In some examples, the performance analyser 18 may distinguish between data relating to a detangling process 110 and data relating to styling. The performance analyser 18 may perform such distinguishing by determining the presence of a drier or by the average trajectory of brush strokes. By distinguishing between the detangling process 110 and the hairstyling process 112, the performance analyser can selectively apply the detangling model to the detangling process data and the force model to the hairstyling data 112.

### User Satisfaction Model

In some examples, the performance analyser 18 may apply a satisfaction model to determine a user happiness, representative of a level of satisfaction of the user with the haircare routine, based on a happiness score received from the face emotion classifier 17. The performance analyser 18 may only analyse the user satisfaction for images pertaining to the end of a haircare session. For example, the performance analyser 17 may receive an output from the brush marker position detecting module 5 or brush motion classifier 16 indicating that the brush marker has been stationary for a threshold time indicating that the haircare routine has finished. By detecting a user's happiness (or lack of) at the end of the haircare session, the face emotion classifier 17 can advantageously determine a user's satisfaction with the routine and provide appropriate feedback. The feedback can include, for example, advice for further styling, product selection advice and positive messaging, such as “you look great today,” to enhance wellbeing, for example.

### Other Performance Analysis Models

The above approaches to performance modelling and analysis could be applied to other significant components of the haircare routine thereby producing further component models. The performance analyser can then apply such models further enriching the feedback personalisation in the a end-user case or providing more sophisticated ways of demonstrating product superiority/in use performance in the study use case.

One further example performance models envisaged as part of this disclosure is a brush stroke model. A brush stroke model could be trained using brush motion parameter data from a plurality of video sequences of users performing a haircare routine with a brush and marker. The training data can be labelled to highlight which routines resulted in healthy hair, an unhappy emotion, numerous detangling events etc. A relative hair health may be quantified according to any of: a level of shine of the hair, a volume of the hair, a number of split ends, a moisture level, a dandruff level etc. A relative level of hair health may be determined manually for the training data or the system may determine the level of health by analysing the images accordingly. The performance analyser 18 can then subsequently monitor user brush strokes using the brush model and provide feedback to the user in relation to the likely outcome of a particular brushing technique. The brush stroke model may receive be categorised according to user hair type/style.

Other models may include a model for grip/brush curling.

### 5. Feedback

Feedback is generated 41 based on the performance analysis and output 42 to the user by one or more of the display 12, audio feedback and haptic feedback, for example.

The at least one item of feedback may comprise at least one of (i) indicating a target hair region for the application of a product or appliance; (ii) indicating a hair region of excess application of a product or appliance. In the example of haircare activities, feedback items may include indicating a target hair region for the application of a product or appliance and/or indicating a hair region of excess application of a product or appliance such as overheating by a hair dryer, curling or straightening appliance.

In some examples, the feedback may be personalised to a particular hair type. The system may receive the hair type as user input or by determining the hair type from one or more images such as the pre- and post-routine images described below. The system may determine the hair type by performing segmentation as described above.

### Immediate Feedback

In some examples, the system 1 may provide feedback to the user during the haircare routine. If the performance analyser 18 detects a specific event, immediate feedback may be given related to that event. For example, if the performance analyser 18 detects a detangling event, the system can provide feedback “at the moment of entanglement.” The feedback may include brushing strategies to deal with pain during detangling, such as gripping the hair with a hand at the root and brushing with the other hand or brushing the tangled hair in short sections starting at the end of the hair, or recommending the immediate application of a formulated product (chemical treatment) to the tangled regions, such as a detangling solution or leave on conditioner. The feedback may identify regions of high entanglement (as illustrated in FIG. 13) for applying the brushing strategies or a recommended product.

In a further example, if the performance analyser 18 detects brush strokes associated with an undesirable outcome using the brush stroke model, the system 1 may provide immediate remedial feedback. The feedback may include advice on a better brushing stroke and may include an animation illustrating such strokes.

In a further example, if the performance analyser 18 applies the force model and detects an inadequate grip between the brush and the hair (pain score greater than a threshold at onset of brush strokes), the system may provide immediate feedback such as providing information on brushing technique (brushing stroke, rotation of brush etc) or suggestions of heat application.

### End of Routine Feedback

In some examples, the system 1 may provide feedback to the user at the completion of the haircare routine. The system 1 may detect the completion of the haircare routine based on the haircare implement remaining static or out of the image boundary for a threshold length of time. In other examples, the user may provide manual input to indicate that the routine has completed.

In some examples, the feedback may include a report summarising the haircare routine. For example, the report may indicate the number and/or location of detangling events detected with the detangling model, statistics on brushing stroke based on the brush stroke model output and/or an average grip between the brush and hair based on the force model output. The data may be illustrated relative to a population distribution of comparable users or relative to similar data captured previously for a different product, enabling the user to attribute changes in performance to the product change. The data may be presented with images captured during the routine, a record of the products used and the user's hair type and condition.

### Multi-Session Feedback

In some examples, the system 1 may provide feedback to the user that relates to an aspect of performance measured over a plurality of sessions. Typically, such an assessment of performance is carried out for the same user across a number of different sessions.

In such examples, analysing the haircare performance comprises determining one or more performance parameters based on:
i) one or more brushing parameters and/or the one or more facial expressions of the user in a current haircare session; and
ii) one or more corresponding brushing parameters and/or one or more facial expressions of the user from a previous personal care session.

By providing multi-session feedback, the system can allow a user to compare their current haircare performance with that in a previous session. For example, feedback could be “Why don't you brush more slowly, like you did this morning”. Alternatively, the change in performance could result in a new chemical treatment recommendation such as “It seems that you have more tangles than usual. Why don't you try applying Product X?”, in which Product X is of a type formulated to reduce hair tangling.

### Types of Feedback

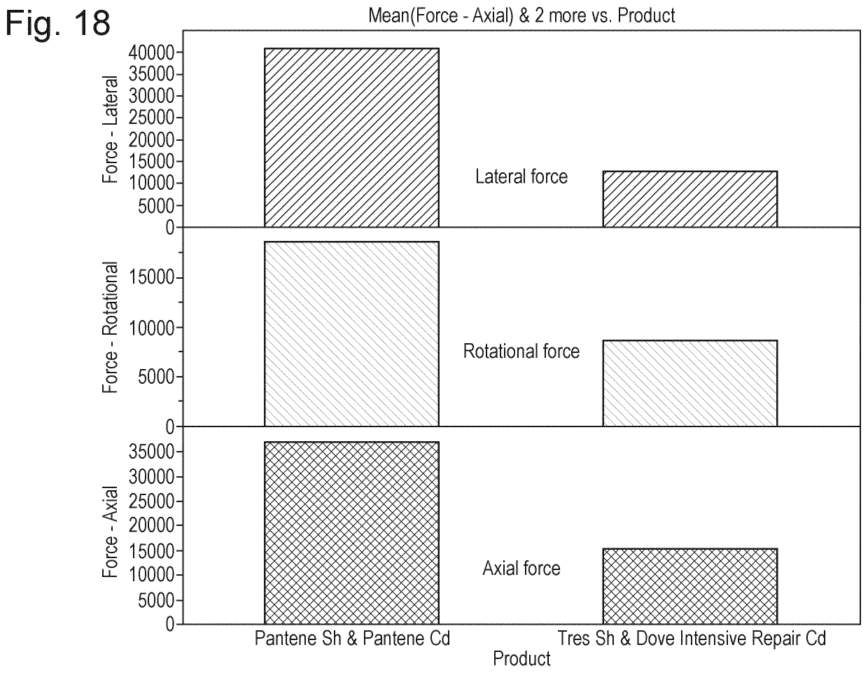

In some examples, the feedback may include product recommendations for use during future hair washing or haircare routines. For example, the product recommendations may relate to formulated products for reducing detangling events, improving brush-hair grip and/or improved hair styling or hair health outcomes. FIG. 18 illustrates that the applied brush force can depend upon formulated product choice. The product recommendations may also include implement recommendations, such as a finer brush, etc.

In some examples, feedback may be provided based on how the user achieves their end style, for example brushing actions during styling and brush-hair grip. In a similar way to the immediate feedback described above, the system 1 may provide feedback in the form of recommended brushing techniques.

In some examples, the system 1 may provide feedback based on user satisfaction from the user satisfaction model, indicating how satisfied the user is with their end look. If the happiness score is less than a happiness threshold, the feedback may highlight differences (brushing style, grip, product choice) between the haircare routine and a previous haircare routine that led to a higher happiness score and provide recommendations for next time. If the happiness score is greater than the happiness threshold, the system may provide feedback in the form of positive messaging to instil confidence and support wellbeing.

### Example Application

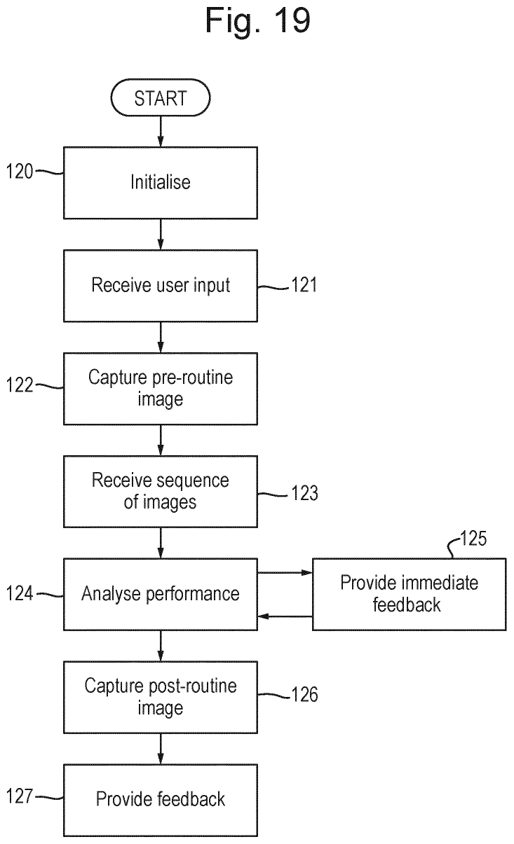

FIG. 19, illustrates an example method of use of the disclosed system. The system 1 may be deployed in a mobile application (app) on a smart phone or tablet or similar personal mobile device.

The system can be advantageously applied in a consumer facing application following user download from an app store or similar. The system may also be advantageously employed in product research and development. For example, users recruited onto a study can use the application and performance analysis data can be used to demonstrate effectiveness of haircare products. For example, the data may quantify the performance of a formulated product in “easing” detangling events (by e.g. reducing the forces needed).

After the user starts the application, the system may perform some initial set up (step 120). For example, the system may enable a camera on the device and provide instructions which guide the user to place the device such that good images of the hairstyling event can be captured. The user can use the device as a mirror at distance guided by instructions from the app (too near, too far, too low, too high etc).

Before commencing the haircare routine, the system may present a number of questions to the user (for example, what is your hair style? How often do you colour your hair? etc) and receive appropriate user input in response (step 121).

The system may capture and store a pre-routine image of the user (step 122).

As the user commences their haircare routine, the system receives a sequence of images from the camera of the device (step 123). The system may then analyse the performance (step 124) as described in detail above. In some examples, the system may analyse the haircare performance by tracking a position of a haircare implement in the sequence of images. In some examples, the system may analyse the haircare performance by analysing a facial expression of the user. The system may analyse the haircare performance by determining one or more performance parameters (e.g. brush-hair grip, brush stroke trajectory) or detecting one or more haircare events (e.g. detangling event). The system may perform different performance analysis depending on a stage of the haircare routine, such as the detangling process and the styling process.

In response to the performance analysis, the system may provide immediate corrective feedback to the user (step 125) as described above.

Following completion of the haircare routine the system may capture and store a post-routine image (step 126). The pre-routine image and post-routine image can be segmented to isolate the hair from the background and then classified against a ‘known’ shape scale. The images can define the user's start and end hair type/style and influence feedback such as product recommendations most suitable for their hair type.

At step 127, the system can provide post-routine feedback as described above.

The brush tracking systems as exemplified above can enable purely visual-based tracking of a brush and facial features. No sensors need be placed on the brush. No sensors need be placed on the person brushing. The technique can be implemented robustly with sufficient performance on currently available mobile phone technologies. The technique can be performed using conventional 2D camera video images.

Throughout the present specification, the expression ‘module’ is intended to encompass a functional system which may comprise computer code being executed on a generic or a custom processor, or a hardware machine implementation of the function, e.g. on an application-specific integrated circuit.

Although the functions of, for example, the face tracking module 4, the brush marker position detecting module 5, the brush marker orientation estimator/detector module 6 and the classifier 10 have been described as distinct modules, the functionality thereof could be combined within a suitable processor as single or multithread processes, or divided differently between different processors and/or processing threads. The functionality can be provided on a single processing device or on a distributed computing platform, e.g. with some processes being implemented on a remote server.

At least part of the functionality of the data processing system may be implemented by way of a smartphone application or other process executing on a mobile telecommunication device. Some or all of the described functionality may be provided on the smartphone. Some of the functionality may be provided by a remote server using the long range communication facilities of the smartphone such as the cellular telephone network and/or wireless internet connection.

It will be appreciated that aspects of the disclosure may be applicable more broadly than in haircare. For example, various embodiments may have applications in personal grooming. The personal grooming activity may comprise one of a toothcare activity, a skin care activity and a haircare activity. The personal grooming activity may comprise tooth brushing and the at least one item of feedback may comprise indicating sufficient or insufficient level of brushing in plural brushing regions of the mouth. The feedback information may comprise giving a visual indication of a level of brushing, on the user's face in a position corresponding to the brushing region.

Other embodiments are intentionally within the scope of the accompanying claims.

Throughout the present specification, the descriptors relating to relative orientation and position, such as “horizontal”, “vertical”, “top”, “bottom” and “side”, are used in the sense of the orientation of the apparatus as presented in the drawings. However, such descriptors are not intended to be in any way limiting to an intended use of the described or claimed invention. Further, reference herein to the determination of a force may relate to the determination of a proxy for force, such as acceleration for example.

It will be appreciated that any reference to “close to”, “before”, “shortly before”, “after” “shortly after”, “higher than”, or “lower than”, etc, can refer to the parameter in question being less than or greater than a threshold value, or between two threshold values, depending upon the context.

1. A computer-implemented method for generating feedback for a user performing haircare, the computer-implemented method comprising:
receiving a sequence of images of a head of the user and a haircare implement, the haircare implement being a hairstyling implement;
determining one or more implement motion parameters by tracking a position or an orientation of the haircare implement using the sequence of images;
determining a detangling event if one or more of: linear velocity, angular velocity, linear acceleration, and angular acceleration of the haircare implement is less than a corresponding detangling motion threshold, the detangling event being an event in which tangles from hair of the user are being removed; and
generating feedback for providing to the user based on the determined detangling event.

2. The computer-implemented method of claim 1, wherein tracking the position or the orientation of the haircare implement comprises tracking the position or the orientation relative to the head of the user.

3. The computer-implemented method of claim 1, wherein tracking the position or the orientation of the haircare implement comprises tracking a fiducial marker coupled to an end of the haircare implement.

4. The computer-implemented method of claim 3, wherein the fiducial marker comprises a substantially spherical marker having a plurality of coloured quadrants disposed around a longitudinal axis corresponding to a longitudinal axis of the haircare implement.

5. The computer-implemented method of claim 1, wherein the one or more implement motion parameters include one or more of: implement position; implement orientation; linear velocity; angular velocity; linear acceleration; angular acceleration; and implement path.

6. The computer-implemented method of claim 1, wherein the haircare implement comprises a hairbrush.

7. The computer-implemented method of claim 1, further comprising determining a detangling event if one or more of: linear velocity, angular velocity, liner acceleration and angular acceleration are less than a corresponding detangling motion threshold.

8. The computer-implemented method of claim 1, further comprising determining an inadequate implement stroke based on an implement path.

9. The computer-implemented method of claim 1, further comprising:
identifying a suitable chemical treatment in accordance with a detected performance parameter or haircare event;
wherein generating the feedback comprises generating instructions to the user to perform the identified chemical treatment.

10. The computer-implemented method of claim 1, wherein generating the feedback comprises generating instructions for the user to operate the haircare implement in a particular way.

11. The computer-implemented method of claim 1, further comprising:
identifying an implement technique in accordance with a detected performance parameter or haircare event;
wherein generating the feedback comprises generating instructions to the user to perform the implement technique.

12. The computer-implemented method of claim 10, further comprising:
determining a facial expression of the user in the sequence of images; and
determining the detected performance parameter based on the facial expression of the user and the one or more implement motion parameters;
wherein the implement technique is identified in accordance with the detected performance parameter.

13. A computer program product comprising computer readable instructions which, when executed on a computer, causes the computer to perform the computer-implemented method of claim 1.

14. A haircare monitoring system comprising a processor configured to perform the computer-implemented method of claim 1.

15. A method comprising:
receiving, by a processor, an image of a user;
detecting, by the processor, a face of the user in the image;
determining, by the processor, a first location of a first face landmark in the image after detecting the face;
classifying, by the processor, an emotion of the user based on the first location;
identifying, by the processor, a portion of the image including a feature of a marker of a haircare implement;
determining, by the processor, an orientation of the haircare implement based on the portion of the image;
determining, by the processor, a position of the haircare implement based on the portion of the image;
determining, by the processor, a brushing parameter based on the first location, the orientation, and the position;
analysing, by the processor, a haircare performance of the user based on the emotion and the brushing parameter; and
providing, by the processor, feedback to the user based on the haircare performance.

16. The method of claim 15, further comprising determining, by the processor, a second location of a second face landmark in the image after detecting the face.

17. The method of claim 16, wherein:
the first face landmark is associated with a nose of the user; and
the second face landmark is associated with the nose of the user, a corner of a head of the user, a centre of the head, a corner of one of at least one eye of the user, or a centre of one of the at least one eye.

18. The method of claim 15, wherein analysing, by the processor, the haircare performance comprises detecting, by the processor, a detangling event based on the emotion and the brushing parameter.

19. The method of claim 15, wherein the brushing parameter comprises at least one of: a linear speed of the haircare implement, an angular speed of the haircare implement, a velocity of the haircare implement, or an acceleration of the haircare implement.

20. The method of claim 15, wherein the feature comprises at least one of: a shape of the marker or a colour pattern of the marker.
